(19) United States
(12) Patent Application Publication
(10) Pub. No.: US 2022/0257753 A1
(43) Pub. Date: Aug. 18, 2022

(54) Vaccine Adjuvants for Antigen Delivery

(71) Applicant: Bobban Subhadra, Sarasota, FL (US)

(72) Inventor: Bobban Subhadra, Sarasota, FL (US)

(21) Appl. No.: 17/669,191

(22) Filed: Feb. 10, 2022

Related U.S. Application Data
(60) Provisional application No. 63/148,792, filed on Feb. 12, 2021.

(51) Int. Cl. A61K 39/39; A61K 39/00; A61K 39/12; A61K 39/02; A61P 37/04; A61P 31/16

(52) U.S. Cl. CPC A61K 39/39; A61K 2039/521; A61K 39/12; A61K 39/02; A61P 37/04; A61P 31/16; A61K 2039/6087; A61K 2039/55511; A61K 2039/627; A61K 2039/64; A61K 2039/55583; A61K 2039/55577; A61K 2039/55505; A61K 2039/5252; A61K 39/0011

(57) ABSTRACT

Cellulose nanoparticle formulations, containing cellulose nanocrystals or nanofibrils, for use as vaccine adjuvants and/or as antigen delivery systems, and the use of the adjuvant formulations in immunogenic and vaccine compositions with different antigens. Cellulose nanoparticle formulations demonstrate enhancements in humoral and cellular immunogenicity of vaccine antigens, particularly subunit vaccine antigens, when utilized alone or in combination with immunostimulatory agents. Further identification of physical and chemical properties of the cellulose nanoparticle formulations can be manipulated to enhance antigen efficiency and adjuvant tolerability in vivo. Relating to the use of the formulations in the treatment of diseases of humans and animals.

### FIELD OF THE INVENTION

The present invention relates to vaccine adjuvants. More specifically, the present invention is an immunological composition comprising one or more antigens and cellulose nanoparticles stabilized in a nanoemulsion system.

### BACKGROUND OF THE INVENTION

Bacterial, viral and parasitic infections are a common occurrence in humans and animals. Diseases caused by these infectious agents are often resistant to antimicrobial pharmaceutical therapies, leaving little to no effective treatment options. Consequently, vaccinological approaches have been increasingly adopted to mediate infectious diseases. One of the more common approaches utilizes the whole infectious agent, which may be suitable for use in a vaccine preparation after chemical inactivation or appropriate genetic manipulation. Alternatively, the protein subunit of the pathogen can be expressed in a recombinant expression system and purified for use in a vaccine preparation as well. However, both of those methods have proven to be less effective at stimulating an immune response and thus a need for an improved vaccination method is apparent. Despite the success of currently approved adjuvants, there remains a need for improved adjuvants and delivery systems that enhance protective antibody responses, especially in populations that respond poorly to current vaccines.

### BRIEF DESCRIPTION OF THE DRAWINGS

FIG. 1 depicts (a) a schematic representation of cellulose nanoparticle (CNP) in two dimensions, (b) a representation of CNP in three dimensions, and (c) a preformed CNP under phase contrast darkfield microscope;

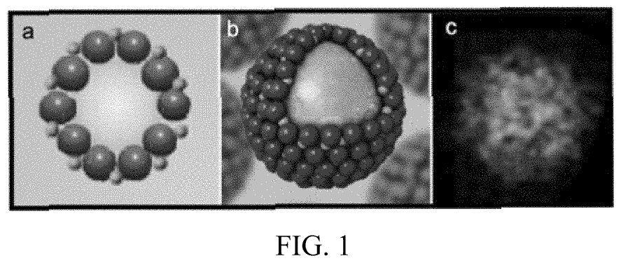

FIG. 2 depicts nanocellulose fibrils under scanning electron microscope;

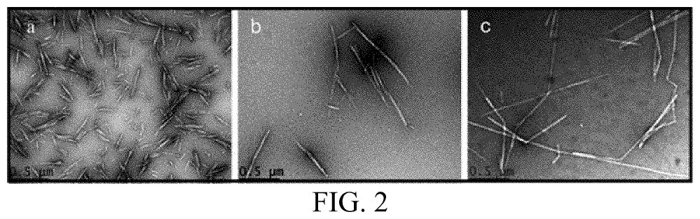

FIG. 3 depicts confocal laser scanning microscopy images of CNP emulsions stabilized by cellulose nanocrystals (CNC) containing increasing amounts of hexadecane stained with 4,4-difluoro-4-bora-3a,4a-diaza-s-indacene from (a) the original 10/90 oil/water Pickering emulsion and (b) 65% of internal phase images to form a three-dimensional reconstruction;

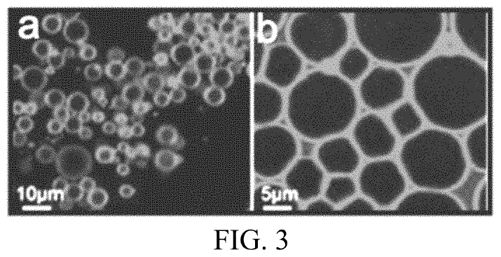

FIG. 4 depicts dark-field microscopy images of oil-in-water CNP nanoemulsions stabilized by (a) nanofibrillated cellulose (NFC) and (b) CNC water-in-oil emulsions stabilized by (c) NFC and oil-in-water-in-oil double emulsions stabilized by (d) NFC/NFCC12;

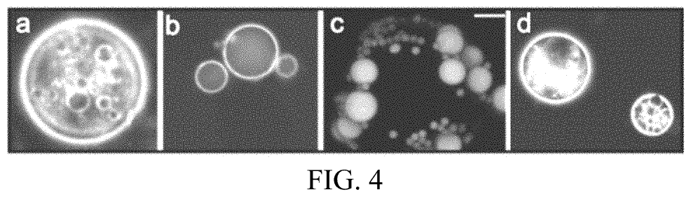

FIG. 5 depicts images showing a representative droplet distribution one week after the preparation of hexadecane-in-water Pickering emulsions stabilized by different cellulose nanocrystal sources and concentrations: 1.5 g L1 ;2g L1 and 5 g L1 for CCN (top), bacterial cellulose (BCN) (middle) and Cladophora (ClaCN) (bottom) with scale bar at 10 mm;

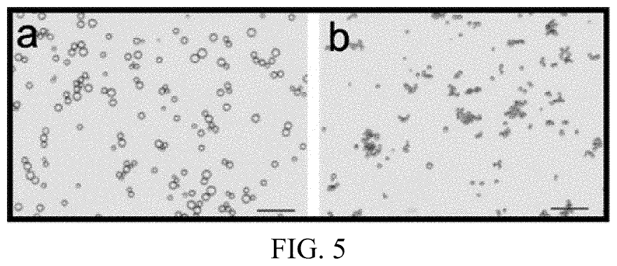

FIG. 6 depicts images showing a representative CNP droplet distribution one week after the preparation of squalene-in-water nanoemulsions stabilized by different cellulose nanocrystal sources and concentrations: 1.5 g L1; 2 g L1 and 5 g L1 for CCN (top), BCN (middle) and ClaCN (bottom) with scale bar at 10 mm;

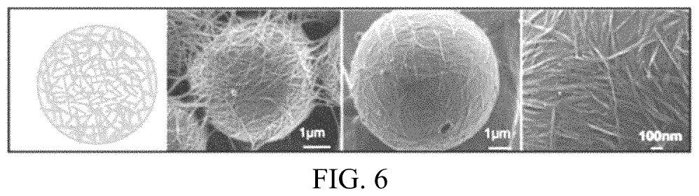

FIG. 7 depicts schematic showing a CNP nanoemulsion adjuvant system with enhanced cell delivery of an antigen, increased contact of the emulsion droplet with the cell surface, due to shape deformation, facilitates antigen-antibody binding at the interface, boosting cellular internalization of the emulsion droplets;

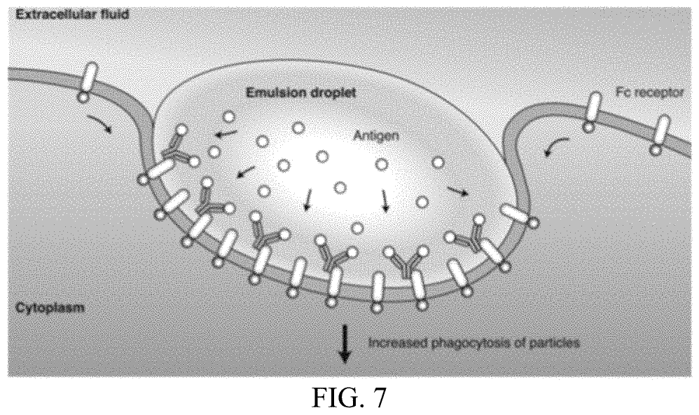

FIG. 8 depicts confocal laser scanning microscopy (CLSM) micrographs of CNP oil-in-water-in-oil double emulsions: (a) CNC (b) cellulose nanofibrils (CNF), and water was stained with fluorescein and hexadecane was stained with boron-dipyrromethene (BODIPY) 665/676 with scale bars 20 μm in (a, b);

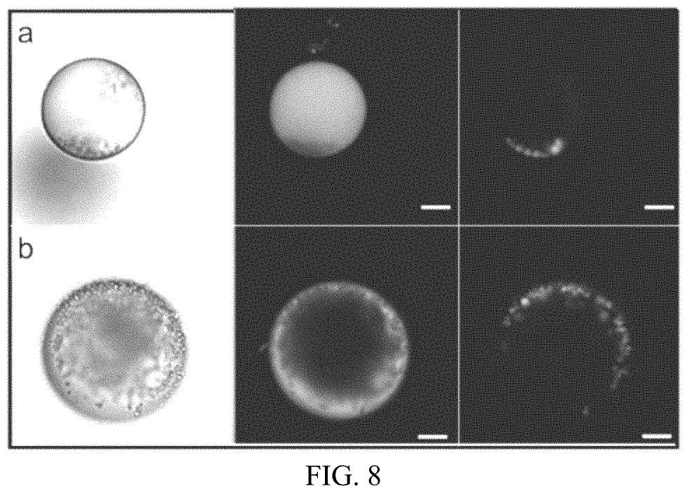

FIG. 9 depicts CNP enhanced antigen uptake and the intracellular fate of antigens, specifically, (a1-3) shows the process of the phagocytosis of Pickering emulsion adjuvant system (PPAS)/ovalbumin (OVA) complexes by bone marrow-derived dendritic cells (BMDCs), and (b) shows intracellular lysosome formation (b1), lysosome escape (b2) and cytosolic antigen delivery (b3);

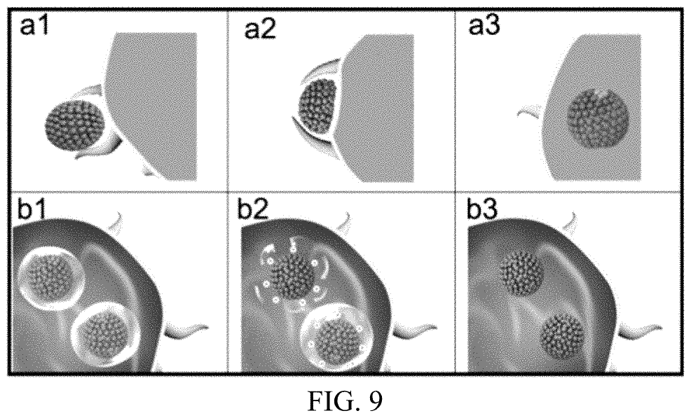

FIG. 10 depicts CNP functions as a potent adjuvant for Influenza A virus subtype H1N1 vaccination, (a-d) shows H1N1 vaccinations, C57BL/6 mice (nD6) were administered with the indicated formulations at prime-boost manner (2-week interval), and challenged by influenza virus (A/FM/1/47) on day 28 (50 times the LD50 per mouse), (a) shows serum HA-specific IgG titres over time, (b) shows serum haemagglutination inhibition (HI) titres 14 days after boost vaccination, (c) shows enzyme-linked immune absorbent spot (ELISPOT) analysis of interferon (IFN)- and interleukin (IL)-4 spot-forming cells among splenocytes after ex vivo restimulation with hemagglutinin (HA) on day 35, and (d) shows survival rate after virus challenge;

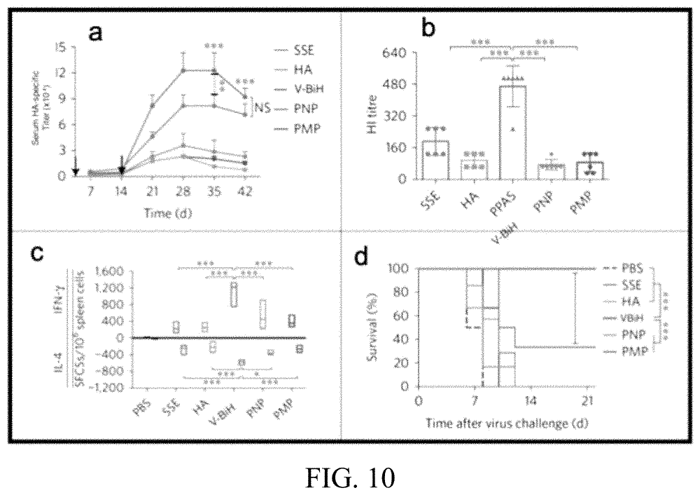

FIG. 11 depicts several different adjuvants that are used in vaccines; and

FIG. 12 depicts formula 1 VaxxiBi CNP formulation for viral adjuvant system.

### DETAILED DESCRIPTIONS OF THE INVENTION

All illustrations of the drawings are for the purpose of describing selected versions of the present invention and are not intended to limit the scope of the present invention.

The present disclosure relates to vaccine adjuvants and antigen delivery systems. More specifically, the present disclosure is an immunological composition comprising one or more antigens and cellulose nanoparticles stabilized in a nanoemulsion system. The term “adjuvant” generally refers to any substance that enhances the humoral or cellular immune response to an antigen. Adjuvants are used for two purposes: They slow down the release of antigens from the injection site and they enhance the stimulation of the immune system. Conventional vaccines generally consist of a crude preparation of inactivated, or killed, or modified live pathogens. Impurities associated with these cultures of pathological microorganisms can act as an adjuvant to enhance the immune response. However, the immunity that is elicited by vaccines that use homogeneous preparations of pathological microorganisms or purified protein subunits as antigens is often weak. Therefore, the addition of some exogenous substances such as an adjuvant becomes necessary. In addition, in some cases, synthetic and subunit vaccines can be expensive to manufacture. Further, in some cases, the pathogen may not be commercially grown, and thus synthetic/subunit vaccines are the only viable option. The addition of an adjuvant may allow lower doses of antigen to be used to stimulate a similar immune response, thereby reducing the cost of producing the vaccine. Thus, the effectiveness of some injectable drugs can be significantly increased when the agent is combined with an adjuvant.

The nanoemulsion adjuvant system of the present disclosure comprises a combination of surface modified or unmodified nanocellulose, lipids with other lipid components such as Polyethylene glycol (PEG)-lipids and optionally non-cationic lipids; the cellulose nanoparticles can be used as vaccine adjuvants and antigen delivery systems; and the lipid nanoparticles can be used in combination with other immunostimulatory compounds. Additionally, the nanoemulsion adjuvant system can further comprise Lipid nanoparticles (CNPs), an adjuvant and an immunogen, and pharmaceutical formulations comprising the CNPs adjuvant or compositions of the present disclosure in a pharmaceutically acceptable carrier. CNPs constitute an alternative to other particulate systems, such as emulsions, liposomes, micelles, microparticles and/or polymeric nanoparticles, for the delivery of active ingredients, such as oligonucleotides and small molecule pharmaceuticals. CNPs and their use for the delivery of oligonucleotides have been previously disclosed. Lipid-based nanoparticles as pharmaceutical drug carriers have also been disclosed. Further provided are methods of producing an immune response against an immunogen in a subject comprising: administering the immunogen and a CNPs adjuvant of the present disclosure to the subject.

Many vaccination regimens exist which allow the manipulation of the type of immune response required for protection from a given pathogen. The use of adjuvants or compounds co-administered with an antigen which augments antigen-specific immune responses have proven to be extremely beneficial for the induction of protective immunity. Many infectious agents rely on mucosal surfaces for entry into the body. Therefore, adjuvants capable of inducing immune responses and which interfere with the early stages of pathogen entry at mucosal surfaces represent powerful tools in the fight against mucosal infections.

Many factors must be considered when choosing an adjuvant though. The adjuvant should cause a relatively low rate of release and absorption of the antigen in an efficient manner with minimal toxic, allergenic, irritant, and other undesirable adverse reactions to the host. To be acceptable, an adjuvant must be non-virucidal, biodegradable, capable of consistently generating high levels of immunity, capable of inducing cross-protection, compatible with many antigens, effective in many species, non-toxic and otherwise safe for the host (e.g., not cause any reactions at the injection site). Other desirable characteristics of an adjuvant are that it is capable of micro-dosing, is moderate dosage, has excellent storage stability, can be dried, can be made without oil, can exist either as a solid or liquid, is isotonic, easy to manufacture, and is inexpensive to receive. Finally, it is imperative that an adjuvant be adjustable, thus inducing either a humoral or cellular immune response, or both, depending on the requirements of the vaccination scenario. However, unfortunately, the number of adjuvants available that satisfy the above requirements are very limited.

The choice of an adjuvant depends on the needs for the vaccine, whether it is increasing the magnitude or function of the antibody response, increasing the cell-mediated immune response, inducing mucosal immunity, or decreasing the dose of antigen. Several adjuvants have been proposed, however, none have been shown to be ideally suited to all types of vaccines. The first adjuvant reported in the literature was Freund's Complete Adjuvant (FCA), which contains a water-in-oil emulsion and mycobacterial extracts. Unfortunately, FCA is poorly tolerated and can lead to uncontrolled inflammation. As more than 80 years have passed since the discovery of FCA, efforts continue to be made to reduce the unwanted side effects of adjuvants with little to no success.

Some other materials that are used as adjuvants include metal oxides (e.g., aluminum hydroxide), potassium alumina, inorganic salt chelates, gelatins, various paraffin-type oils, synthesized resins, alginates, mucoid and polysaccharide compounds, caseinates, and blood-derived substances such as fibrin clots. While these materials are generally effective in stimulating the immune system, none have been found to be completely satisfactory due to host side effects (e.g., sterile abscess, organ damage, carcinogenicity, or allergic reactions) or undesirable pharmaceutical properties (for example, rapid resorption or poor resorption control from the injection site, or material edema).

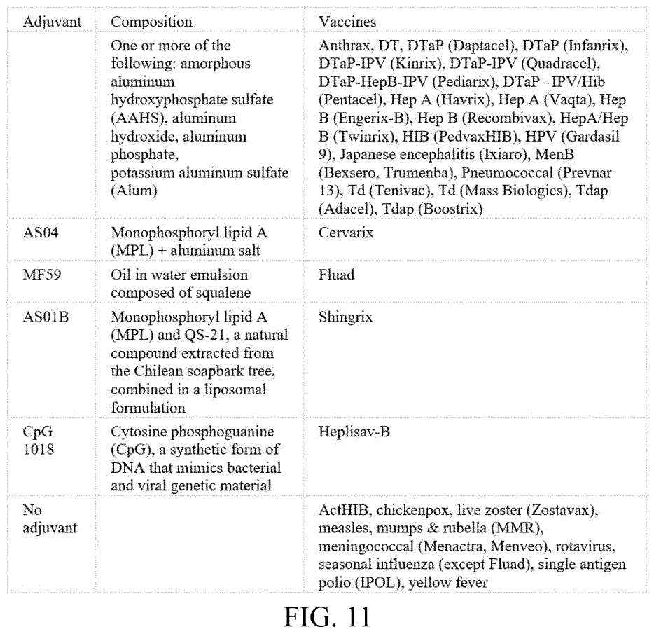

FIG. 11 shows several different adjuvants that are used in various vaccines.

The present invention focuses on the exploitation of the pliability (mechanosensing deformation) and mobility of antigen delivery systems may confer an effective strategy to elicit robust prophylactic and therapeutic immune responses.

Accordingly, as a first aspect, the present invention provides a method of producing an immune response against an immunogen in a subject, comprising: administering the immunogen to the subject in an immunogenically effective amount; and administering an alphavirus adjuvant to the subject in an adjuvant effective amount, wherein the alphavirus adjuvant does not express the immunogen.

In an embodiment of the present invention, the lipid nanoparticle further comprises an immunostimulatory agent selected from saponin, squalene, aluminum phosphate and aluminum hydroxide.

In an embodiment of the present invention, at least one of the one or more antigens can be selected from antigens from RSV, Chlamydia, Dengue, CMV, Ebola, Varicella, Herpes viruses, HIV, or Influenza. In certain aspects of these embodiments, the antigens are subunit antigens. The one or more antigens may be physically encapsulated in the CNP before or after CNP preparation. The one or more antigens may be adsorbed, covalently coupled, ionically-interacted, or formulated onto surfaces of the CNP adjuvant.

The immunological compositions of the present invention can be in the form of an aerosol, dispersion, solution, or suspension. The immunological compositions can be formulated for intramuscular, oral, sublingual, buccal, parenteral, nasal, subcutaneous, intradermal, or topical administration.

The present invention is also directed to methods of immunizing a subject or treating or preventing various diseases or disorders in the subject by administering to the subject an effective amount of the immunological compositions of the present invention.

The present invention is also directed to methods of immunizing a subject or treating or preventing various diseases or disorders in the subject by co-administering to the subject 1) an effective amount of the CNP of the present invention and 2) i) an agonist selected from a TLR agonist and a STING agonist; and/or ii) an antigen.

The present invention is directed to immunological compositions comprising one or more antigens and a lipid nanoparticle (CNP) containing cationic lipids or ionizable cationic lipids. Such compositions can be used as vaccine adjuvants or vaccine antigen delivery agents, preferably for subunit vaccines. CNP formulations described herein demonstrate enhancements in humoral and/or cellular immunogenicity of vaccine antigens, for example, subunit vaccine antigens, when utilized alone or in combination with immunostimulatory agents (e g , small molecule or oligonucleotide TLR agonists or STING agonists). In certain embodiments, the present invention provides co-formulation of CNP systems, with or without immunostimulatory agents, with peptide or protein antigens as vaccines.

Without being bound by any theory, an advantage of this co-formulation strategy is that it is believed to enable maintenance of the antigen dose in close proximity to the adjuvant at the administration site, thereby reducing rapid dispersion of active agents, leading to enhanced immune response and potential reduction of systemic adverse effects. The present invention further identifies physical and chemical properties of these CNPs which lead to enhanced antigen efficiency and adjuvant tolerability in vivo.

CNPs, when appropriately designed, were shown to improve the delivery efficiency of antigens, e.g., subunit antigens, to target cells, enable combination and co-delivery of antigens and adjuvants, and facilitate the intracellular delivery of antigens to better potentiate desirable intracellular immune responses. The CNPs were shown to be potent vaccine adjuvants, capable of inducing strong antibody and T cell responses in preclinical rodent models when combined with recombinant protein antigens for a number of tested antigens including Dengue and HBV. As illustrated by the examples, robust adjuvant activity was demonstrated for a synthetic immunostimulatory oligonucleotides (IMO 2125 as described in Agrawal et al., 2007, Biochem Soc Trans. 35(Pt 6):1461-7) and antigens (HBsAg and DEN-80) in vitro and in vivo. Furthermore, the T cell response had a strong CD8 component, which was superior to that induced by other adjuvants tested, such as aluminum-based adjuvant and monophosphoryl lipid A and was of a magnitude typically only seen with live virus vaccines.

The CNP adjuvants described herein offer the potential for a number of significant advantages over existing adjuvant technologies. Potential advantages include enabling modulation of the adaptive immune response to produce more effective type of immunity (e.g., Th1/Th2) for specific antigens, yielding improved antibody titers and cell-mediated immunity, broadening responses, reducing antigen dose and/or number of doses, and enabling immunization of patients with weakened immune systems.

In a particular embodiment, the size of the CNPs ranges between about 1 and 1000 nm, preferably between about 10 and 500 nm, and more preferably between about 100 to 200 nm.

Various Antigenic Components That Can Be Used with CNP System:

The disclosed compositions and methods are applicable to a wide variety of antigens. In certain embodiments, the antigen is a protein (including recombinant proteins), polypeptide, or peptide (including synthetic peptides). In certain embodiments, the antigen is a lipid or a carbohydrate (polysaccharide). In certain embodiments, the antigen is a protein extract, cell (including tumor cell), or tissue. The compositions provided herein can contain one or more antigens (e.g., at least two, three, four, five, or six antigens).

In specific embodiments, antigens can be selected from the group consisting of the following: (a) polypeptides suitable to induce an immune response against cancer cells; (b) polypeptides suitable to induce an immune response against infectious diseases; (c) polypeptides suitable to induce an immune response against allergens; and (d) polypeptides suitable to induce an immune response in farm animals or pets.

In certain embodiments, the compositions of the present invention can be used in combination with an immunoregulatory therapy to target either activating receptors or inhibitory receptors. See, e.g., Mellman et al., 2013, Nature 480:480-489. The immunoregulatory therapy can be, for example, a T cell engaging agent selected from agonistic antibodies which bind to human OX40, to GITR, to CD27, or to 4-IBB, and T-cell bispecific antibodies (e.g. T cell-engaging BiTE™ antibodies CD3-CD19, CD3-EpCam, CD3-EGFR), IL-2 (Proleukin), Interferon (IFN) alpha, antagonizing antibodies which bind to human CTLA-4 (e.g. ipilimumab), to PD-1, to PD-L1, to TIM-3, to BTLA, to VISTA, to LAG-3, or to CD25.

Examples of antigens or antigenic determinants include the following: the RSV F or G antigens, Chlamydia antigens such as the Major outer membrane protein (mOMP), the Dengue type 1 to 4 envelope proteins, the HIV antigens gp140 and gp160; the influenza antigens hemagglutinin, M2 protein, and neuraminidase; hepatitis B surface antigen or core; and circumsporozoite protein of malaria, or fragments thereof.

Appropriate antigens for use with this CNP technology may be derived from, but not limited to, pathogenic bacterial, fungal, or viral organisms, Streptococcus species, Candida species, Brucella species, Salmonella species, Shigella species, Pseudomonas species, Bordetella species, Clostridium species, Norwalk virus, Bacillus anthracis, Mycobacterium tuberculosis, human immunodeficiency virus (HIV), Chlamydia species, human Papillomaviruses, Influenza virus, Paramyxovirus species, Herpes virus, Cytomegalovirus, Varicella-Zoster virus, Epstein-Barr virus, Hepatitis viruses, Plasmodium species, Trichomonas species, Ebola, sexually transmitted disease agents, viral encephalitis agents, protozoan disease agents, fungal disease agents, cancer cells, or mixtures thereof. Other appropriate molecules incorporated in the nanoparticle vaccines may include self-antigens, adhesins, or surface exposed cell signaling receptors or ligands. A variety of diseases and disorders may be treated by such nanoparticle vaccine constructs or assemblies, including inflammatory diseases, infectious diseases, cancer, genetic disorders, organ transplant rejection, autoimmune diseases and immunological disorders.

By activating antigen presenting cells (APCs) including dendritic cells (DCs) and macrophages, adjuvants hold the potential to unleash the natural functions of cytotoxic T lymphocytes (CTLs) to kill pathogens or cancer. Many adjuvants including Toll-like receptor (TLR) agonists engage innate immune receptors on APCs, inducing APCs to present antigens, produce cytokines, and provide costimulatory signals to antigen-specific CD8 T cells. In response to these signals, CD8 T cells proliferate and differentiate into CTLs capable of killing infected or tumor cells expressing their target antigen. In addition, such signals activate CD4 T cells, inducing their expansion and differentiation into Th1 or Th2 T helper cells.

One of the most important targets of improved adjuvant technology lies in the field of cancer immunotherapy, where the adaptive immune system is exploited to kill cancer cells based on their expression of cancer-associated antigens or neoantigens. The effectiveness of cancer immunotherapy depends on the generation and activation of tumor-specific CTLs and on their maintenance of activity in vivo, leading to killing of tumor cells and a long-lasting antitumor memory response. Thus, immune checkpoint inhibitors such as anti-PD-1, anti-PD-L1, and anti-CTLA-4 have achieved remarkable clinical success in the treatment of melanoma through their action in blocking pathways that inhibit CTL activation. However, even among those tumors known to be susceptible to checkpoint blockade, response rates of only ˜20% have been reported for PD-1/PD-L1 antibody treatment, possibly due to insufficient numbers or activation of tumor-reactive CTLs or their failure to infiltrate tumors. These deficiencies may be exacerbated by immunosuppression induced by the cancer environment. Cancer vaccines targeted to tumor neoantigens can boost the success of immune checkpoint inhibition for cancer treatment by increasing the number and activation of tumor-specific CTLs capable of responding to checkpoint inhibitors. However, the type and magnitude of the T cell response to immunization depends critically on the vaccine adjuvant; currently, only few adjuvants are approved for use in humans. In one embodiment nanocellulose based adjuvant engages and activates human and mouse TLR1/TLR2 heterodimers for antiPD-1/PD-L1 adjuvant immunotherapy.

### Antigens and Diseases:

Compositions can contain one or more antigens., selected from the group consisting of viruses (inactivated, attenuated and modified live), bacteria, parasites, nucleotides (including, without limitation, antigens on nucleic acid-based, e.g. vaccine DNA), polynucleotides, peptides, polypeptides that can be isolated from an organism, recombinant proteins, synthetic peptides, protein extracts, cells (including tumor cells), tissues, polysaccharides, carbohydrates, fatty acids, teichoic acid, peptidoglycans, lipids or glycolipids, immunogenic fragments of nucleotides, individually or in any combination.

Two or more antigens can be combined to provide a multivalent composition that can protect a subject from a wide variety of diseases caused by pathogenic microorganisms.

The amount of antigen that is used to induce an immune response will vary greatly depending on the antigen used, the subject, and the level of response desired, and can be determined by one of ordinary skill in the art. For vaccines containing modified live viruses or attenuated viruses, the therapeutically effective amount of antigen will generally range from about 10 2 tissue culture infectious dose (TCID) 50 to about 10 10 TCID 50, inclusively. For many such viruses, the therapeutically effective dose is generally in the range from about 10 2 TCID 50 to about 10 8 TCID 50, inclusively. In some embodiments, the therapeutically effective dose ranges are from about 10 3 TCID 50 to about 10 6 TCID 50, inclusively. In other embodiments, the therapeutically effective dose ranges are from about 10 4 TCID 50 to about 10 5 TCID 50, inclusively.

For vaccines containing inactivated viruses, the therapeutically effective amount of antigen is generally at least about 100 relative units per dose and is often in the range of about 1,000 to about 4,500 relative units per dose, inclusive. In other embodiments, the therapeutically effective amount of antigen ranges from about 250 to about 4000 relative units per dose, including from about 500 to about 3000 relative units per dose, including from about 750 to about 2000 relative units per dose, including, or from about 1000 to about 1500 relative units per dose, inclusive.

The therapeutically effective amount of antigen in vaccines containing inactivated viruses can also be measured in terms of relative potency (RP) per ml. A therapeutically effective amount is often in the range of about 0.1 to about 50 RP per ml, inclusive. In other embodiments, the therapeutically effective amount of antigen ranges from about 0.5 to about 30 RP per ml, including from about 1 to about 25 RP per ml, including from about 2 to about 20 RP per ml, including, from about 3 to about 15 RPs per ml, including, or from about 5 to about 10 RPs per ml, inclusive.

The number of cells for bacterial antigen that is introduced into the vaccine ranges from about 1×10 6 to about 5×10 10 colony forming units (CFU) per dose, inclusive. In other embodiments, implementation, the number of cells is in the range from about 1×10 7 to 5×10 10 CFU/dose, including, or from about 1×10 8 to 5×10 10 CFU/dose, inclusive. In still other embodiments, the number of cells ranges from about 1×10 2 to 5×10 10 CFU/dose, including, or from about 1×10 4 to 5×10 9 CFU/dose, including, or from about 1×10 5 to 5×10 9 CFU/dose, including, or from about 1×10 6 to 5×10 9 CFU/dose, including, or from about 1×10 6 to 5×10 8 CFU/dose, including, or from about 1×10 7 to 5×10 9 CFU/dose, inclusive.

The number of cells for the parasitic antigen that is introduced into the vaccine ranges from about 1×10 2 to about 1×10 10 per dose, inclusive. In other embodiments, the number of cells ranges from about 1×10 3 to about 1×10 9 per dose, including, or from about 1×10 4 to about 1×10 8 per dose, including, or from about 1×10 5 to about 1×10 7 per dose, including, or from about 1×10 6 to about 1×10 8 per dose, inclusive.

It has surprisingly been found that with the adjuvant compositions described herein, approximately equal amounts of inactivated virus and modified live virus stimulate similar levels of serological response. In addition, small amounts of modified live, attenuated and inactivated virus are needed with the adjuvants described herein as compared to conventional adjuvants to achieve the same level of serological response. These unexpected results demonstrate resource conservation and cost savings in the preparation of immunogenic and vaccine compositions. For vaccines with widespread use, millions of doses per year are required, so these savings can be substantial.

In another embodiment, antigens associated with infection or infectious disease are associated with any of the infectious agents provided herein. In one embodiment, the infectious agent is a virus of the Adenoviridae, Picornaviridae, Herpesviridae, Hepadnaviridae, Flaviviridae, Retroviridae, Orthomyxoviridae, Paramyxoviridae, Papillomaviridae, Rhabdoviridae, Togaviridae or Paroviridae family. In still another embodiment, the infectious agent is adenovirus, coxsackievirus, hepatitis A virus, poliovirus, Rhinovirus, Herpes simplex virus, Varicella-zoster virus, Epstein-barr virus, Human cytomegalovirus, Human herpesvirus, Hepatitis B virus, Hepatitis C virus, yellow fever virus, dengue virus, West Nile virus, HIV, Influenza virus, Measles virus, Mumps virus, Parainfluenza virus, Respiratory syncytial virus, Human metapneumovirus, Human papillomavirus, Rabies virus, Rubella virus, Human bocarivus or Parvovirus B19. In yet another embodiment, the infectious agent is a bacteria of the Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema Vibrio or Yersinia genus. In a further embodiment, the infectious agent is Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycobacterium ulcerans, Mycoplasma pneumoniae, Neisseria gonorrhoeae, Neisseria meningitides, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae or Yersinia pestis. In another embodiment, the infectious agent is a fungus of the Candida, Aspergillus, Cryptococcus, Histoplasma, Pneumocystis or Stachybotrys genus. In still another embodiment, the infectious agent is C. albicans, Aspergillus fumigatus, Aspergillus flavus, Cryptococcus neoformans, Cryptococcus laurentii, Cryptococcus albidus, Cryptococcus gattii, Histoplasma capsulatum, Pneumocystis jirovecii or Stachybotrys chartarum.

In yet another embodiment, the antigen associated with infection or infectious disease is one that comprises VI, VII, E1A, E3-19K, 52K, VP1, surface antigen, 3A protein, capsid protein, nucleocapsid, surface projection, transmembrane proteins, UL6, UL18, UL35, UL38, UL19, early antigen, capsid antigen, Pp65, gB, p52, latent nuclear antigen-1, NS3, envelope protein, envelope protein E2 domain, gp120, p24, lipopeptides Gag (17-35), Gag (253-284), Nef (66-97), Nef (116-145), Pol (325-355), neuraminidase, nucleocapsid protein, matrix protein, phosphoprotein, fusion protein, hemagglutinin, hemagglutinin-neuraminidase, glycoprotein, E6, E7, envelope lipoprotein or non-structural protein (NS). In another embodiment, the antigen comprises pertussis toxin (PT), filamentous hemagglutinin (FHA), pertactin (PRN), fimbriae (FIM 2/3), VlsE; DbpA, OspA, Hia, PrpA, MltA, L7/L12, D15, 0187, VirJ, Mdh, AfuA, L7/L12, out membrane protein, LPS, antigen type A, antigen type B, antigen type C, antigen type D, antigen type E, FliC, FliD, Cwp84, alpha-toxin, theta-toxin, fructose 1,6-biphosphate-aldolase (FBA), glyceraldehydes-3-phosphate dehydrogenase (GPD), pyruvate:ferredoxin oxidoreductase (PFOR), elongation factor-G (EF-G), hypothetical protein (HP), T toxin, Toxoid antigen, capsular polysaccharide, Protein D, Mip, nucleoprotein (NP), RD1, PE35, PPE68, EsxA, EsxB, RD9, EsxV, Hsp70, lipopolysaccharide, surface antigen, Sp1, Sp2, Sp3, Glycerophosphodiester Phosphodiesterase, outer membrane protein, chaperone-usher protein, capsular protein (F1) or V protein. In yet another embodiment, the antigen is one that comprises capsular glycoprotein, Yps3P, Hsp60, Major surface protein, MsgC1, MsgC3, MsgC8, MsgC9 or SchS34.

### EXAMPLE 1

### CNP-HINI Antigen Promotes Humoral and Cellular Immune Responses

After verifying the in vivo safety of CNP and the endotoxin levels of the formulations, CNP -induced antigen-specific immune responses were then investigated in triplicate and similar results were obtained. Notably, CNP maintained high titres of serum OVA-specific IgG over time. Comparison of cytokine release profiles also indicated potent Th1- and Th2-mediated responses. Furthermore, CNP elicited Th1-polarized responses as shown by a IgG2a/IgG1 ratio>1. As a prerequisite for cellular immunity, the proportion of IFN-_secreting CD8C and SINFEKL-MHC IC CD8CT cells increased by 304% and 278%, respectively.

To further assess the immune protections, mice were subsequently challenged by E.G7/OVA lymphoma cells 14 days after the prime-boost immunization. Of all mice injected with CNP (nD6), the average tumor volume was considerably smaller than that in the other groups at all time points. The significantly higher survival rates supported the notion that CNP functioned as a potent adjuvant for enhanced immune protection. The therapeutic efficacy of CNP towards established tumors was next assessed. Mice were inoculated with 106 E.G7/OVA lymphoma cells and vaccinated with the indicated formulations on days 4, 11 and 18. CNP significantly delayed the onset of tumor growth and maintained evidently high survival rates (5/8) in recipients compared to other groups, emphasizing the potential of CNP for therapeutic vaccinations.

Immune protection by H1N1 vaccine with CNP as adjuvant encouraged by the adjuvant activity with OVA, CNP in clinically relevant vaccinations was further employed. To estimate the performance for prophylactic vaccinations, C57BL/6 mice were vaccinated with influenza HA. Notably, significantly higher levels of HA-specific IgG secretion and HI titers were detected in the serum.

In addition, CNP also induced robust cellular immune reactions with a distinctly higher population of IFN- and IL-4-secreting cells , as well as HA-specific cytotoxic T lymphocytes (CTLs). Then, memory T cell dynamics in response to various formulations were probed. With significantly more memory T cells (CD44C CD62LC) within the CD4C and CD8C T cell population (P <0.001, analysed by one-way ANOVA), CNP was demonstrated to maintain immune memory against re-infection.

Subsequently, the immunized mice were challenged with an intranasal lethal dose of influenza (A/FM/1/47, H1N1) on day 28. CNP evidently increased the survival of mice and rapidly restored their body weight within seven days was observed. Collectively, the Pickering emulsion potentiated the antigen-specific adaptive immune responses and conferred potent protection against infections.

### EXAMPLE 2

### Therapeutic Formulation in the Present Invention

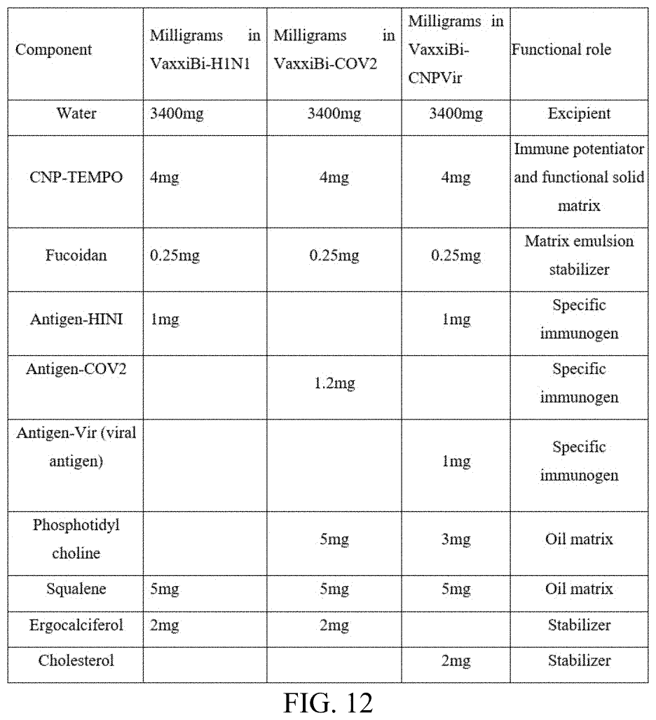

Therapeutic formulation of actives with CNP adjuvant actives are called VaxxiBi. FIG. 12 shows the formula 1 VaxxiBi CNP formulation for a viral adjuvant system.

Although the invention has been explained in relation to its preferred embodiment, it is to be understood that many other possible modifications and variations can be made without departing from the spirit and scope of the invention.

What is claimed is:

1. A therapeutic immunogenic composition comprising:
an adjuvant formulation;
a therapeutically effective amount of antigen component;
the adjuvant formulation comprising a nanocellulose, a sulfated polysaccharide, purified water, and a lipid;
the antigen component selected from the group consisting of inactivated virus, viral proteins, inactivated bacteria, bacterial antigenic proteins, and oncologic tumor antigen;
the antigen component is chemically conjugated to the nanocellulose via chemical conjugation;
the nanocellulose is in a nanofibril form;
the sulfated polysaccharide is fucoidan, sulfated rhamnose, sulfated galactan, ulvans, carrageenan, heparin, and sulfated glycosaminoglycan; and
the lipid is phosphatidyl choline, phosphotidyl ethanolamine, and squalene.

2. The therapeutic immunogenic composition of claim 1, wherein the adjuvant formulation further comprises:
a sterol;
the sterol is in a nanoemulsion system;
the sulfated polysaccharide further comprises heparan sulfates;
the nanocellulose is in a nanocrystal form; and
the sterol is selected from the group consisting of p-sitosterol, stigmasterol, ergosterol, ergocalciferol, and cholesterol.

3. The therapeutic immunogenic composition of claim 1, wherein the antigen component further comprises:
a neoantigen component;
the neoantigen component comprising tumor-reactive cytotoxic lymphocytes and a repetitive array in the nanocellulose matrix.

4. The therapeutic immunogenic composition of claim 2, where in the antigen is chemically conjugated to form the nanocellulose in a repetitive array using 1-cyano-4-dimethylaminopyridinium.

5. The therapeutic immunogenic composition of claim 4, wherein the adjuvant formulation further comprises:
the nanocellulose present in an amount of 0.5 μg to 5,000 μg per dose;
the sulfated polysaccharide is present in an amount of 0.25 μg to 2,000 μg per dose;
the sterol present in an amount of 1 μg to 5,000 μg per dose; and
the lipid present in an amount of 0.01% volume to volume to 50% volume to volume.

6. The therapeutic immunogenic composition of claim 4, wherein the nanocellulose is 10 nm to 300 nm in size.

7. The therapeutic immunogenic composition of claim 6, wherein the surface of the nanocellulose is modified from the group consisting of 2,6,6-tetramethylpiperidine 1-oxyl radical-oxidized, 3-aminopropylphosphoric acid-functionalized cellulose nanofibrils, (2,2,6,6-tetramethylpiperidin-1-yl)oxyl-oxidized, and cationic surface modifiers.

8. The therapeutic immunogenic composition of claim 6, further comprising an immunostimulatory agent selected from the group consisting of saponin, squalene, aluminum phosphate, and aluminum hydroxide.

9. A therapeutic immunogenic composition comprising:
an adjuvant formulation;
a therapeutically effective amount of antigen component;
the therapeutically effective amount of antigen component comprising a relative potency in the range of at least 0.1 relative potency per milliliter to at least 50 relative potency per milliliter;
the adjuvant formulation comprising purified water, a nanocellulose, a sulfated polysaccharide, and a lipid;
the antigen component being a bacterial antigen component;
the bacterial antigen component comprising a repetitive array in a nanocellulose matrix;
the nanocellulose is in nanofibril form;
the sulfated polysaccharide is fucoidan, sulfated rhamnose, sulfated galactan, ulvans, carrageenan, heparin, and sulfated glycosaminoglycan; and
the lipid is phosphatidyl choline, phosphotidyl ethanolamine, and squalene; and
the antigen component is chemically conjugated to the nanocellulose via chemical conjugation.

10. The therapeutic immunogenic composition of claim 9, wherein the antigen component further comprises:
a viral antigen component;
the viral antigen component comprising a repetitive array in the nanocellulose matrix.

11. The therapeutic immunogenic composition of claim 9, further comprising an immunostimulatory agent selected from the group consisting of saponin, alpha tocopherol, squalene, aluminum phosphate, murabutide and aluminum hydroxide.

12. The therapeutic immunogenic composition of claim 9, wherein the antigen component further comprises:
a neoantigen component;
the neoantigen component comprising tumor-reactive cytotoxic lymphocytes and a repetitive array in the nanocellulose matrix.

13. The therapeutic immunogenic composition of claim 12, further comprising at least one agonists selected from the group consisting of Toll-like receptor agonists and Stimulator of Interferon Gene agonists.

14. The therapeutic immunogenic composition of claim 12, wherein the adjuvant formulation further comprises sterol in a nanoemulsion system;
the sulfated polysaccharide is heparan sulfates;
the nanocellulose is in a nanocrystal form; and
the sterol is selected from the group consisting of p-sitosterol, stigmasterol, ergosterol, ergocalciferol, and cholesterol.

15. The therapeutic immunogenic composition of claim 14, where in the antigen is chemically conjugated to form the nanocellulose in a repetitive array using 1-cyano-4-dimethylaminopyridinium.

16. The therapeutic immunogenic composition of claim 15, wherein the adjuvant formulation further comprises:
the nanocellulose present in an amount of 0.5 μg to 5,000 μg per dose;
the sulfated polysaccharide is present in an amount of 0.25 μg to 2,000 μg per dose;
the sterol present in an amount of 1 μg to 5,000 μg per dose; and
the lipid present in an amount of 0.01% volume to volume to 50% volume to volume.

17. The therapeutic immunogenic composition of claim 16, wherein the nanocellulose is 10 nm to 300 nm in size.

18. The therapeutic immunogenic composition of claim 16, wherein the surface of the nanocellulose is modified from the group consisting of 2,6,6-tetramethylpiperidine 1-oxyl radical-oxidized, 3-aminopropylphosphoric acid-functionalized cellulose nanofibrils, (2,2,6,6-tetramethylpiperidin-1-yl)oxyl-oxidized, and cationic surface modifiers.

19. The therapeutic immunogenic composition of claim 16, wherein the therapeutically effective amount of antigen component contains a relative potency in the range of at least 0.5 relative potency per milliliter to at least 30 relative potency per milliliter.

20. The therapeutic immunogenic composition of claim 16, further comprising an immunostimulatory agent selected from the group consisting of saponin, squalene, aluminum phosphate, and aluminum hydroxide.
